(19) United States
(12) Patent Application Publication
(10) Pub. No.: US 2021/0262952 A1
(43) Pub. Date: Aug. 26, 2021

(54) METHODS FOR IMAGING USING X-RAY FLUORESCENCE

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 17/236,653

(22) Filed: Apr. 21, 2021

Related U.S. Application Data
(63) Continuation of application No. PCT/CN2018/114125, filed on Nov. 6, 2018.

(51) Int. Cl. G01N 23/223; G01N 23/04; G06T 7/00; G06T 15/08

(52) U.S. Cl. CPC G01N 23/223; G01N 23/043; G06T 7/0012; G06T 2207/10121; G01N 2223/076; G06T 2207/10081; G06T 15/08

(57) ABSTRACT

Disclosed herein is a method comprising: causing emission of characteristic X-rays of a chemical element introduced into a human body; capturing images of a portion of the human body with the characteristic X-rays; determining a three-dimensional distribution of the chemical element in the portion based on the images.

### BACKGROUND

X-ray fluorescence (XRF) is the emission of characteristic X-rays from a material that has been excited by, for example, exposure to high-energy X-rays or gamma rays. An electron on an inner orbital of an atom may be ejected, leaving a vacancy on the inner orbital, if the atom is exposed to X-rays or gamma rays with photon energy greater than the ionization potential of the electron. When an electron on an outer orbital of the atom relaxes to fill the vacancy on the inner orbital, an X-ray (fluorescent X-ray or secondary X-ray) is emitted. The emitted X-ray has a photon energy equal the energy difference between the outer orbital and inner orbital electrons.

For a given atom, the number of possible relaxations is limited. As shown in FIG. 1A, when an electron on the L orbital relaxes to fill a vacancy on the K orbital (L→K), the fluorescent X-ray is called Kα. The fluorescent X-ray from M→K relaxation is called Kβ. As shown in FIG. 1B, the fluorescent X-ray from M→L relaxation is called La, and so on.

### SUMMARY

Disclosed herein is a method comprising: causing emission of characteristic X-rays of a chemical element introduced into a human body; capturing images of a portion of the human body with the characteristic X-rays; determining a three-dimensional distribution of the chemical element in the portion based on the images.

According to an embodiment, the images are captured respectively at multiple locations relative to the human body.

According to an embodiment, the images are captured with a detector configured to move to the multiple locations.

According to an embodiment, the chemical element has an atomic number of 60 or larger.

According to an embodiment, the chemical element is W or Pb.

According to an embodiment, the chemical element is not radioactive.

According to an embodiment, the chemical element is in a chemical compound.

According to an embodiment, causing emission of the characteristic X-rays comprises irradiating the portion with radiation that causes the emission of the characteristic X-rays.

According to an embodiment, the radiation is X-ray or gamma ray.

According to an embodiment, the chemical element is introduced into the human body through the bloodstream of the human body.

According to an embodiment, the images are captured with a detector with an X-ray absorption layer configured to absorb the characteristic X-rays, wherein the X-ray absorption layer comprises Ge.

According to an embodiment, the X-ray absorption layer comprises Li.

According to an embodiment, the detector comprise a cooler configured to cool the X-ray absorption layer below 80 K.

According to an embodiment, the detector comprises an array of pixels, and is configured to count numbers of photons of the characteristic X-rays incident on the pixels within a period of time.

According to an embodiment, the detector is configured to count the numbers of X-ray photons within a same period of time.

According to an embodiment, the pixels are configured to operate in parallel.

According to an embodiment, each of the pixels is configured to measure its dark current.

According to an embodiment, the detector further comprises a collimator configured to limit fields of view of the pixels.

According to an embodiment, the detector does not comprise a scintillator.

According to an embodiment, energies of particles of the radiation are above 40 keV.

According to an embodiment, capturing the images comprises counting numbers of photons of the characteristic X-rays within a period of time.

According to an embodiment, the X-ray absorption layer comprises an electrode; wherein the detector comprises: a first voltage comparator configured to compare a voltage of the electrode to a first threshold, a second voltage comparator configured to compare the voltage to a second threshold, a counter configured to register a number of X-ray photons reaching the X-ray absorption layer, and a controller; wherein the controller is configured to start a time delay from a time at which the first voltage comparator determines that an absolute value of the voltage equals or exceeds an absolute value of the first threshold; wherein the controller is configured to activate the second voltage comparator during the time delay; wherein the controller is configured to cause the number registered by the counter to increase by one, if the second voltage comparator determines that an absolute value of the voltage equals or exceeds an absolute value of the second threshold.

According to an embodiment, the detector further comprises an integrator electrically connected to the electrode, wherein the integrator is configured to collect charge carriers from the electrode.

According to an embodiment, the controller is configured to activate the second voltage comparator at a beginning or expiration of the time delay.

According to an embodiment, the detector further comprises a voltmeter, wherein the controller is configured to cause the voltmeter to measure the voltage upon expiration of the time delay.

According to an embodiment, the controller is configured to determine an X-ray photon energy based on a value of the voltage measured upon expiration of the time delay.

According to an embodiment, the controller is configured to connect the electrode to an electrical ground.

According to an embodiment, a rate of change of the voltage is substantially zero at expiration of the time delay.

According to an embodiment, a rate of change of the voltage is substantially non-zero at expiration of the time delay.

### BRIEF DESCRIPTION OF FIGURES

FIG. 1A and FIG. 1B schematically show mechanisms of XRF.

FIG. 2 shows a flowchart for a method, according to an embodiment.

FIG. 3 schematically shows a system, according to an embodiment.

FIG. 4 schematically shows an X-ray detector of the system, according to an embodiment.

FIG. 5A-FIG. 5C each schematically show a cross-sectional view of the X-ray detector, according to an embodiment.

FIG. 6A-FIG. 6B each schematically show a component diagram of an electronic system of the X-ray detector, according to an embodiment.

FIG. 7 schematically shows a temporal change of an electric current caused by charge carriers generated by an incident photon of X-ray, and a corresponding temporal change of a voltage, according to an embodiment.

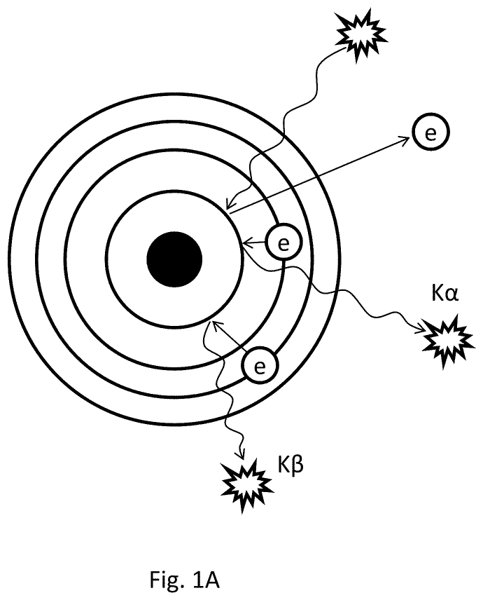

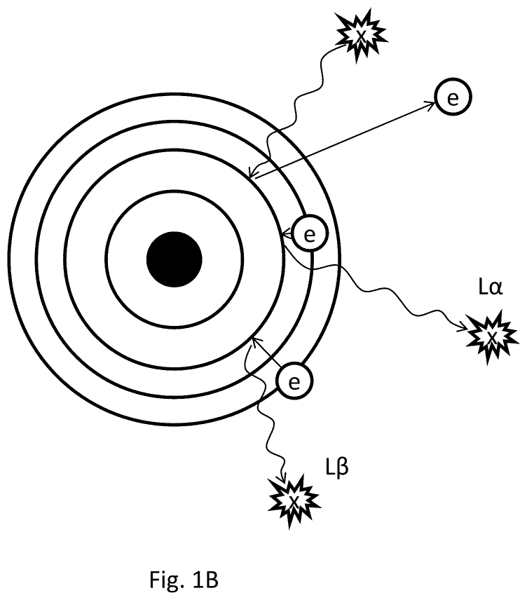

### DETAILED DESCRIPTION

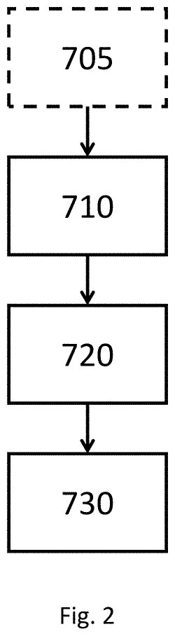

FIG. 2 shows a flowchart for a method, according to an embodiment. In optional procedure 705, a chemical element is introduced into a human body. The chemical element may be a non-radioactive chemical element. The chemical element is not necessarily a pure element but can be in a chemical compound. For example, the chemical element may have ligands attached thereto. The chemical element may be introduced into the human body orally in pills or liquids, or by injection into muscles or the blood stream. Examples of the chemical element may include tungsten (W), lead (Pb), and chemical elements with an atomic number of 60 or larger. In procedure 710, emission of the characteristic X-rays of the chemical element introduced into the human body is caused, for example, by irradiating a portion of the human body with radiation (e.g., high-energy X-rays or gamma rays) that causes the emission of the characteristic X-rays. In procedure 720, images of the portion of the human body are captured with the characteristic X-rays. The images may be captured respectively at multiple locations relative to the human body. In procedure 730, a three-dimensional distribution of the chemical element in the portion of the human body is determined based on the images.

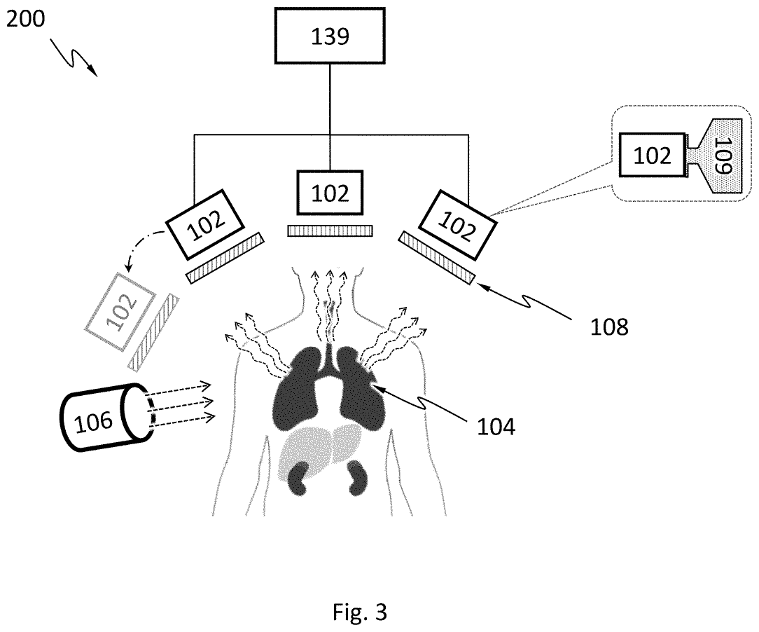

FIG. 3 schematically shows a system 200. The system 200 includes one or more X-ray detectors 102, according to an embodiment. The X-ray detectors 102 may be positioned at or moved to multiple locations relative to an object 104 (e.g., a portion of a human body). For example, the X-ray detectors 102 may be at multiple locations along a semicircle around the portion of the human body or along the length of the portion of the human body. The X-ray detectors 102 may be arranged at about the same distance or different distances from the object 104. Other suitable arrangement of the X-ray detectors 102 may be possible. The X-ray detectors 102 may be spaced equally or unequally apart in the angular direction. The positions of the X-ray detectors 102 are not necessarily fixed. For example, some of the X-ray detectors 102 may be movable towards and away from the object 104 or may be rotatable relative to the object 104. In an embodiment, at least some of the X-ray detector 102 do not comprise a scintillator.

FIG. 3 schematically shows that the system 200 may include a radiation source 106, according to an embodiment. The system 200 may include more than one radiation source. The radiation source 106 irradiates the object 104 with radiation that can cause the chemical element (e.g., tungsten, or lead) to emit characteristic X-rays (e.g., by fluorescence). The chemical element may not be radioactive. The radiation from the radiation source 106 may be X-ray or gamma ray. The energies of the particles of the radiation may be above 40 keV. The radiation source 106 may be movable or stationary relative to the object 104. The X-ray detectors 102 capture images of the object 104 with the characteristic X-rays (e.g., by detecting the intensity distribution of the characteristic X-rays). The X-ray detectors 102 may be disposed at different locations around the object 104 where the X-ray detectors 102 do not receive the radiation from the radiation source 106 that is not scattered by the object 104. As shown in FIG. 3, the X-ray detectors 102 may avoid those positions where they would receive radiation from the radiation source 106 that has passed through the object 104. The X-ray detectors 102 may be movable or stationary relative to the object 104.

The object 104 may be a portion (e.g., the thyroid) of a human body. In an example, non-radioactive chemical element in the form of chemical compound is introduced into the human body and absorbed by the portion. When the radiation from the radiation source 106 is directed toward the portion of the human body, the non-radioactive chemical element inside the portion of the human body is excited by the radiation and emits the characteristic X-rays of the chemical element. The characteristic X-rays may include the K lines, or the K lines and the L lines. The images of the portion of the human body are captured with the characteristic X-rays of the chemical element respectively by X-ray detectors 102 at multiple locations relative to the portion of the human body. The images of the portion of the human body may be captured with X-ray detectors 102 configured to move to multiple locations relative to the portion, as shown in FIG. 3. The X-ray detectors 102 may disregard X-rays with energies different from characteristic X-rays of the chemical element. Spatial (e.g., three-dimensional) distribution of the chemical element inside the portion of the human body may be determined from these images. For example, the system 200 may have a processor 139 configured to determine the three-dimensional distribution of the chemical element in the portion of the human body, based on these images.

FIG. 3 schematically shows that some of the X-ray detectors 102 may further comprise a collimator 108, according to an embodiment. The collimator 108 may be positioned between the object 104 and the X-ray detectors 102. The collimator 108 is configured to limit fields of view of pixels of the X-ray detectors 102. For example, collimator 108 may allow only X-rays with certain angles of incidence to reach the X-ray detectors 102. The range of angles of incidence may be ≤0.04 sr, or ≤0.01 sr. The collimator 108 may be affixed on the X-ray detectors 102 or separated from the X-ray detectors 102. There may be spacing between the collimator 108 and the X-ray detectors 102. The collimator 108 may be movable or stationary relative to the X-ray detectors 102. The system 200 may include more than one collimator 108.

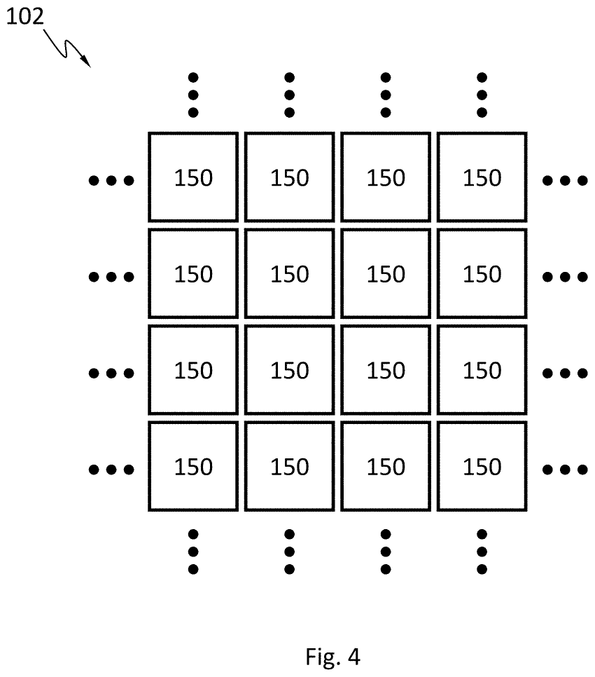

FIG. 4 schematically shows one of the X-ray detectors 102, according to an embodiment. This one X-ray detector 102 has an array of pixels 150. The array may be a rectangular array, a honeycomb array, a hexagonal array or any other suitable array. Each pixel 150 is configured to count numbers of photons of X-rays (e.g., the characteristic X-rays of chemical element) incident on the pixels 150 within a period of time. The pixels 150 may be configured to operate in parallel. For example, when one pixel 150 measures an incident X-ray photon, another pixel 150 may be waiting for an X-ray photon to arrive. The pixels 150 may not have to be individually addressable. Each of the X-ray detectors 102 may be configured to count the numbers of X-ray photons within the same period of time. Therefore, capturing the images of the portion of the human body comprises counting numbers of photons of the characteristic X-rays within a period of time. Each pixel 150 may be able to measure its dark current, such as before or concurrently with receiving each X-ray photon. Each pixel 150 may be configured to deduct the contribution of the dark current from the energy of the X-ray photon incident thereon.

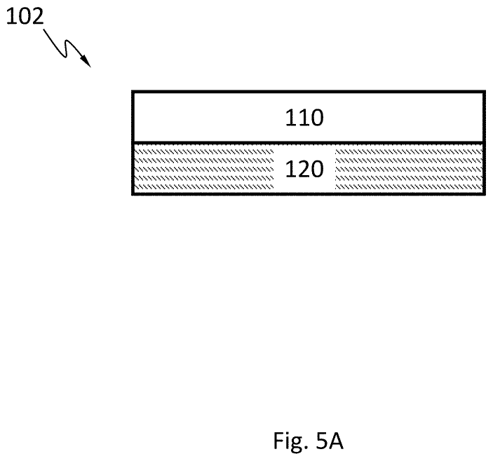

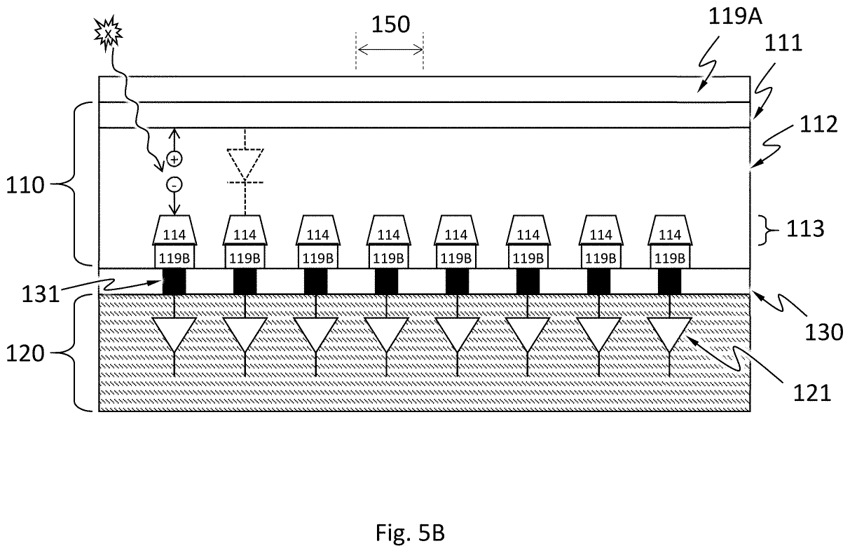

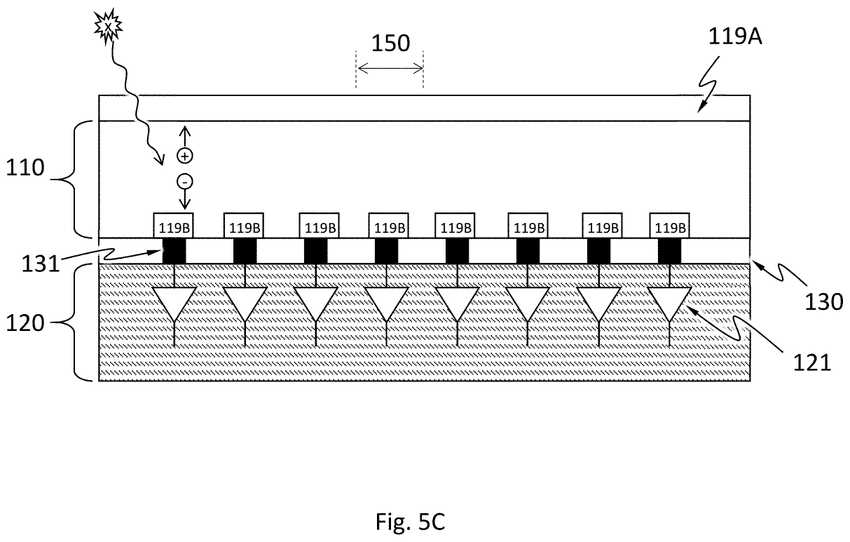

FIG. 5A schematically shows one X-ray detector 102, according to an embodiment. The X-ray detector 102 may include an X-ray absorption layer 110 and an electronics layer 120 (e.g., an ASIC) for processing or analyzing electrical signals incident X-ray generates in the X-ray absorption layer 110. The X-ray absorption layer 110 may be configured to absorb the characteristic X-rays of the chemical element, and may include a semiconductor material such as, germanium (Ge), lithium (Li), or a combination thereof. The semiconductor may have a high mass attenuation coefficient for the characteristic X-rays. The X-ray detector 102 may comprise a cooler 109 (as shown in FIG. 3) configured to cool the X-ray absorption layer below 80 K to reduce electrical noise induced by thermal excitations of valence electrons. The cooler 109 may use liquid nitrogen cooling or pulse tube refrigerator.

As shown in a detailed cross-sectional view of the X-ray detector 102 in FIG. 5B, according to an embodiment, the X-ray absorption layer 110 may include one or more diodes (e.g., p-i-n or p-n) formed by a first doped region 111, one or more discrete regions 114 of a second doped region 113. The second doped region 113 may be separated from the first doped region 111 by an optional the intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example in FIG. 5B, each of the discrete regions 114 of the second doped region 113 forms a diode with the first doped region 111 and the optional intrinsic region 112. Namely, in the example in FIG. 5B, the X-ray absorption layer 110 has a plurality of diodes having the first doped region 111 as a shared electrode. The first doped region 111 may also have discrete portions.

When an X-ray photon hits the X-ray absorption layer 110 including diodes, the X-ray photon may be absorbed and generate one or more charge carriers by a number of mechanisms. An X-ray photon may generate 10 to 100000 charge carriers. The charge carriers may drift to the electrodes of one of the diodes under an electric field. The field may be an external electric field. The electric contact 1198 may include discrete portions each of which is in electric contact with the discrete regions 114.

As shown in an alternative detailed cross-sectional view of the X-ray detector 102 in FIG. 5C, according to an embodiment, the X-ray absorption layer 110 may include a resistor of a semiconductor material such as, germanium (Ge), lithium (Li), or a combination thereof, but does not include a diode. The semiconductor may have a high mass attenuation coefficient for the characteristic X-rays.

When an X-ray photon hits the X-ray absorption layer 110 including a resistor but not diodes, it may be absorbed and generate one or more charge carriers by a number of mechanisms. An X-ray photon may generate 10 to 100000 charge carriers. The charge carriers may drift to the electric contacts 119A and 1198 under an electric field. The field may be an external electric field. The electric contact 1198 includes discrete portions.

The electronics layer 120 may include an electronic system 121, suitable for processing or interpreting signals generated by X-ray photons incident on the X-ray absorption layer 110. The electronic system 121 may include an analog circuitry such as a filter network, amplifiers, integrators, and comparators, or a digital circuitry such as a microprocessor, and memory. The electronic system 121 may include components shared by the pixels or components dedicated to a single pixel. For example, the electronic system 121 may include an amplifier dedicated to each pixel and a microprocessor shared among all the pixels. The electronic system 121 may be electrically connected to the pixels by vias 131. Space among the vias may be filled with a filler material 130, which may increase the mechanical stability of the connection of the electronics layer 120 to the X-ray absorption layer 110. Other bonding techniques are possible to connect the electronic system 121 to the pixels without using vias.

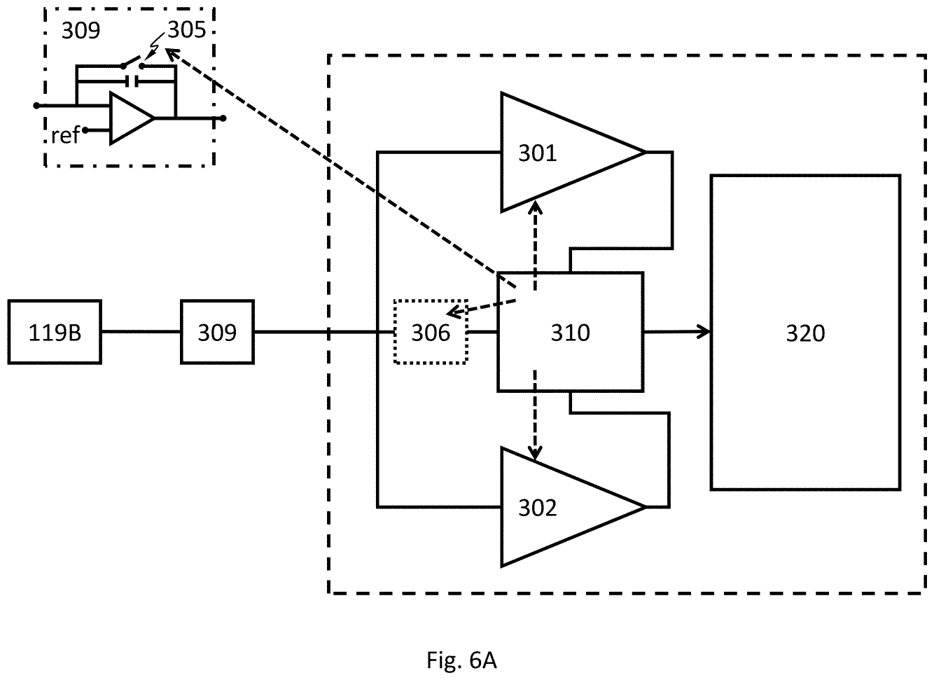

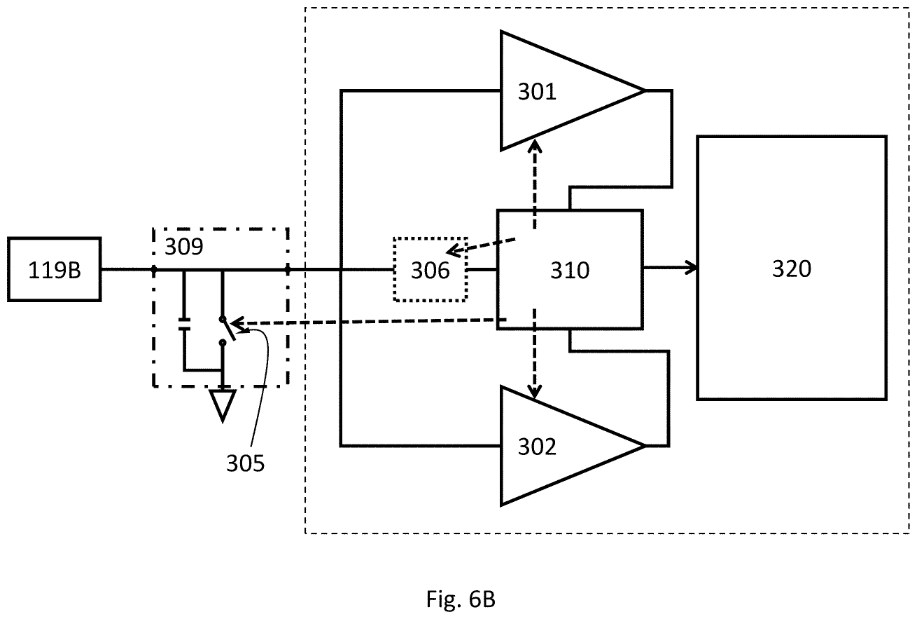

FIG. 6A and FIG. 6B each show a component diagram of the electronic system 121, according to an embodiment. The electronic system 121 may include a first voltage comparator 301, a second voltage comparator 302, a counter 320, a switch 305, an optional voltmeter 306 and a controller 310.

The first voltage comparator 301 is configured to compare the voltage of at least one of the electric contacts 119B to a first threshold. The first voltage comparator 301 may be configured to monitor the voltage directly, or to calculate the voltage by integrating an electric current flowing through the electrical contact 119B over a period of time. The first voltage comparator 301 may be controllably activated or deactivated by the controller 310. The first voltage comparator 301 may be a continuous comparator. Namely, the first voltage comparator 301 may be configured to be activated continuously and monitor the voltage continuously. The first voltage comparator 301 may be a clocked comparator. The first threshold may be 5-10%, 10%-20%, 20-30%, 30-40% or 40-50% of the maximum voltage one incident photon of X-ray may generate on the electric contact 119B. The maximum voltage may depend on the energy of the incident photon of X-ray, the material of the X-ray absorption layer 110, and other factors. For example, the first threshold may be 50 mV, 100 mV, 150 mV, or 200 mV.

The second voltage comparator 302 is configured to compare the voltage to a second threshold. The second voltage comparator 302 may be configured to monitor the voltage directly or calculate the voltage by integrating an electric current flowing through the diode or the electrical contact over a period of time. The second voltage comparator 302 may be a continuous comparator. The second voltage comparator 302 may be controllably activate or deactivated by the controller 310. When the second voltage comparator 302 is deactivated, the power consumption of the second voltage comparator 302 may be less than 1%, less than 5%, less than 10% or less than 20% of the power consumption when the second voltage comparator 302 is activated. The absolute value of the second threshold is greater than the absolute value of the first threshold. As used herein, the term “absolute value” or “modulus” |x| of a real number x is the non-negative value of x without regard to its sign. Namely,

$$\vert x \vert = \{ \begin{matrix}x , if x \geq 0 \\ - x , if x \leq 0\end{matrix} .$$

The second threshold may be 200%-300% of the first threshold. The second threshold may be at least 50% of the maximum voltage one incident photon of X-ray may generate on the electric contact 1198. For example, the second threshold may be 100 mV, 150 mV, 200 mV, 250 mV or 300 mV. The second voltage comparator 302 and the first voltage comparator 310 may be the same component. Namely, the system 121 may have one voltage comparator that can compare a voltage with two different thresholds at different times.

The first voltage comparator 301 or the second voltage comparator 302 may include one or more op-amps or any other suitable circuitry. The first voltage comparator 301 or the second voltage comparator 302 may have a high speed to allow the electronic system 121 to operate under a high flux of incident photons of X-rays. However, having a high speed is often at the cost of power consumption.

The counter 320 is configured to register at least a number of photons of X-rays incident on the pixel 150 encompassing the electric contact 119B. The counter 320 may be a software component (e.g., a number stored in a computer memory) or a hardware component (e.g., a 4017 IC and a 7490 IC).

The controller 310 may be a hardware component such as a microcontroller and a microprocessor. The controller 310 is configured to start a time delay from a time at which the first voltage comparator 301 determines that the absolute value of the voltage equals or exceeds the absolute value of the first threshold (e.g., the absolute value of the voltage increases from below the absolute value of the first threshold to a value equal to or above the absolute value of the first threshold). The absolute value is used here because the voltage may be negative or positive, depending on whether the voltage of the cathode or the anode of the diode or which electrical contact is used. The controller 310 may be configured to keep deactivated the second voltage comparator 302, the counter 320 and any other circuits the operation of the first voltage comparator 301 does not require, before the time at which the first voltage comparator 301 determines that the absolute value of the voltage equals or exceeds the absolute value of the first threshold. The time delay may expire before or after the voltage becomes stable, i.e., the rate of change of the voltage is substantially zero. The phase “the rate of change of the voltage is substantially zero” means that temporal change of the voltage is less than 0.1%/ns. The phase “the rate of change of the voltage is substantially non-zero” means that temporal change of the voltage is at least 0.1%/ns.

The controller 310 may be configured to activate the second voltage comparator during (including the beginning and the expiration) the time delay. In an embodiment, the controller 310 is configured to activate the second voltage comparator at the beginning of the time delay. The term “activate” means causing the component to enter an operational state (e.g., by sending a signal such as a voltage pulse or a logic level, by providing power, etc.). The term “deactivate” means causing the component to enter a non-operational state (e.g., by sending a signal such as a voltage pulse or a logic level, by cut off power, etc.). The operational state may have higher power consumption (e.g., 10 times higher, 100 times higher, 1000 times higher) than the non-operational state. The controller 310 itself may be deactivated until the output of the first voltage comparator 301 activates the controller 310 when the absolute value of the voltage equals or exceeds the absolute value of the first threshold.

The controller 310 may be configured to cause at least one of the number registered by the counter 320 to increase by one, if, during the time delay, the second voltage comparator 302 determines that the absolute value of the voltage equals or exceeds the absolute value of the second threshold.

The controller 310 may be configured to cause the optional voltmeter 306 to measure the voltage upon expiration of the time delay. The controller 310 may be configured to connect the electric contact 119B to an electrical ground, so as to reset the voltage and discharge any charge carriers accumulated on the electric contact 119B. In an embodiment, the electric contact 119B is connected to an electrical ground after the expiration of the time delay. In an embodiment, the electric contact 119B is connected to an electrical ground for a finite reset time period. The controller 310 may connect the electric contact 119B to the electrical ground by controlling the switch 305. The switch may be a transistor such as a field-effect transistor (FET).

In an embodiment, the system 121 has no analog filter network (e.g., a RC network). In an embodiment, the system 121 has no analog circuitry.

The voltmeter 306 may feed the voltage it measures to the controller 310 as an analog or digital signal.

The electronic system 121 may include an integrator 309 electrically connected to the electric contact 119B, wherein the integrator is configured to collect charge carriers from the electric contact 119B. The integrator 309 can include a capacitor in the feedback path of an amplifier. The amplifier configured as such is called a capacitive transimpedance amplifier (CTIA). CTIA has high dynamic range by keeping the amplifier from saturating and improves the signal-to-noise ratio by limiting the bandwidth in the signal path. Charge carriers from the electric contact 119B accumulate on the capacitor over a period of time (“integration period”). After the integration period has expired, the capacitor voltage is sampled and then reset by a reset switch. The integrator 309 can include a capacitor directly connected to the electric contact 119B.

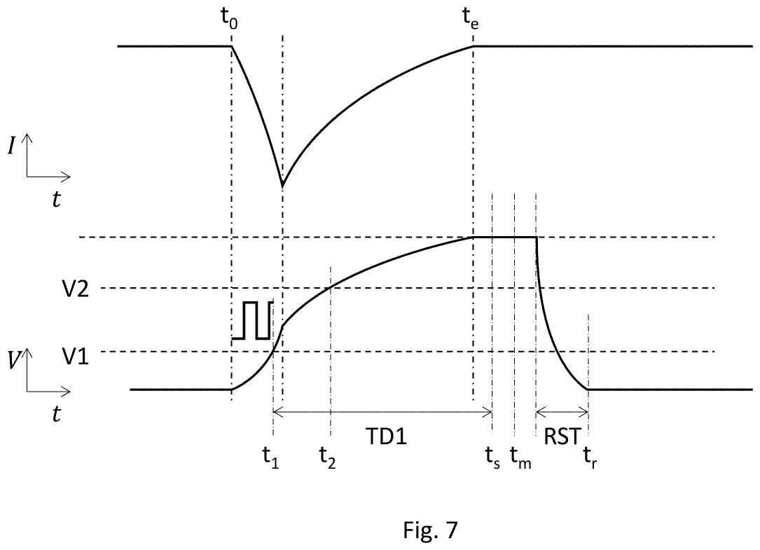

FIG. 7 schematically shows a temporal change of the electric current flowing through the electric contact 119B (upper curve) caused by charge carriers generated by a photon of X-ray incident on the pixel 150 encompassing the electric contact 119B, and a corresponding temporal change of the voltage of the electric contact 119B (lower curve). The voltage may be an integral of the electric current with respect to time. At time to, the photon of X-ray hits pixel 150, charge carriers start being generated in the pixel 150, electric current starts to flow through the electric contact 119B, and the absolute value of the voltage of the electric contact 119B starts to increase. At time t₁, the first voltage comparator 301 determines that the absolute value of the voltage equals or exceeds the absolute value of the first threshold V1, and the controller 310 starts the time delay TD1 and the controller 310 may deactivate the first voltage comparator 301 at the beginning of TD1. If the controller 310 is deactivated before t₁, the controller 310 is activated at t₁. During TD1, the controller 310 activates the second voltage comparator 302. The term “during” a time delay as used here means the beginning and the expiration (i.e., the end) and any time in between. For example, the controller 310 may activate the second voltage comparator 302 at the expiration of TD1. If during TD1, the second voltage comparator 302 determines that the absolute value of the voltage equals or exceeds the absolute value of the second threshold V2 at time t₂, the controller 310 waits for stabilization of the voltage to stabilize. The voltage stabilizes at time tₑ, when all charge carriers generated by the photon of X-ray drift out of the X-ray absorption layer 110. At time tₛ, the time delay TD1 expires. At or after time tₑ, the controller 310 causes the voltmeter 306 to digitize the voltage and determines which bin the energy of the photon of X-ray falls in. The controller 310 then causes the number registered by the counter 320 corresponding to the bin to increase by one. In the example of FIG. 7, time tₛ is after time tₑ; namely TD1 expires after all charge carriers generated by the photon of X-ray drift out of the X-ray absorption layer 110. If time tₑ cannot be easily measured, TD1 can be empirically chosen to allow sufficient time to collect essentially all charge carriers generated by a photon of X-ray but not too long to risk have another incident photon of X-ray. Namely, TD1 can be empirically chosen so that time tₛ is empirically after time tₑ. Time tₛ is not necessarily after time tₑ because the controller 310 may disregard TD1 once V2 is reached and wait for time tₑ. The rate of change of the difference between the voltage and the contribution to the voltage by the dark current is thus substantially zero at tₑ. The controller 310 may be configured to deactivate the second voltage comparator 302 at expiration of TD1 or at t₂, or any time in between.

The voltage at time tₑ is proportional to the amount of charge carriers generated by the photon of X-ray, which relates to the energy of the photon of X-ray. The controller 310 may be configured to determine the energy of the photon of X-ray, using the voltmeter 306.

After TD1 expires or digitization by the voltmeter 306, whichever later, the controller 310 connects the electric contact 119B to an electric ground for a reset period RST to allow charge carriers accumulated on the electric contact 119B to flow to the ground and reset the voltage. After RST, the electronic system 121 is ready to detect another incident photon of X-ray. If the first voltage comparator 301 has been deactivated, the controller 310 can activate it at any time before RST expires. If the controller 310 has been deactivated, it may be activated before RST expires.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method comprising:
causing emission of characteristic X-rays of a chemical element introduced into a human body;
capturing images of a portion of the human body with the characteristic X-rays;
determining a three-dimensional distribution of the chemical element in the portion based on the images.

2. The method of claim 1, wherein the images are captured respectively at multiple locations relative to the human body.

3. The method of claim 2, wherein the images are captured with a detector configured to move to the multiple locations.

4. The method of claim 1, wherein the chemical element has an atomic number of 60 or larger.

5. The method of claim 1, wherein the chemical element is W or Pb.

6. The method of claim 1, wherein the chemical element is not radioactive.

7. The method of claim 1, wherein the chemical element is in a chemical compound.

8. The method of claim 1, wherein causing emission of the characteristic X-rays comprises irradiating the portion with radiation that causes the emission of the characteristic X-rays.

9. The method of claim 8, wherein the radiation is X-ray or gamma ray.

10. The method of claim 1, wherein the chemical element is introduced into the human body through the bloodstream of the human body.

11. The method of claim 1, wherein the images are captured with a detector with an X-ray absorption layer configured to absorb the characteristic X-rays, wherein the X-ray absorption layer comprises Ge.

12. The method of claim 11, wherein the X-ray absorption layer comprises Li.

13. The method of claim 11, wherein the detector comprise a cooler configured to cool the X-ray absorption layer below 80 K.

14. The method of claim 11, wherein the detector comprises an array of pixels, and is configured to count numbers of photons of the characteristic X-rays incident on the pixels within a period of time.

15. The method of claim 14, wherein the detector is configured to count the numbers of X-ray photons within a same period of time.

16. The method of claim 14, wherein the pixels are configured to operate in parallel.

17. The method of claim 14, wherein each of the pixels is configured to measure its dark current.

18. The method of claim 14, wherein the detector further comprises a collimator configured to limit fields of view of the pixels.

19. The method of claim 11, wherein the detector does not comprise a scintillator.

20. The method of claim 8, wherein energies of particles of the radiation are above 40 keV.

21. The method of claim 1, wherein capturing the images comprises counting numbers of photons of the characteristic X-rays within a period of time.

22. The method of claim 11, wherein the X-ray absorption layer comprises an electrode;
wherein the detector comprises:
a first voltage comparator configured to compare a voltage of the electrode to a first threshold,
a second voltage comparator configured to compare the voltage to a second threshold,
a counter configured to register a number of X-ray photons reaching the X-ray absorption layer, and
a controller;
wherein the controller is configured to start a time delay from a time at which the first voltage comparator determines that an absolute value of the voltage equals or exceeds an absolute value of the first threshold;
wherein the controller is configured to activate the second voltage comparator during the time delay;
wherein the controller is configured to cause the number registered by the counter to increase by one, if the second voltage comparator determines that an absolute value of the voltage equals or exceeds an absolute value of the second threshold.

23. The method of claim 22, wherein the detector further comprises an integrator electrically connected to the electrode, wherein the integrator is configured to collect charge carriers from the electrode.

24. The method of claim 22, wherein the controller is configured to activate the second voltage comparator at a beginning or expiration of the time delay.

25. The method of claim 22, wherein the detector further comprises a voltmeter, wherein the controller is configured to cause the voltmeter to measure the voltage upon expiration of the time delay.

26. The method of claim 22, wherein the controller is configured to determine an X-ray photon energy based on a value of the voltage measured upon expiration of the time delay.

27. The method of claim 22, wherein the controller is configured to connect the electrode to an electrical ground.

28. The method of claim 22, wherein a rate of change of the voltage is substantially zero at expiration of the time delay.

29. The method of claim 22, wherein a rate of change of the voltage is substantially non-zero at expiration of the time delay.
